(19) United States
(12) Patent Application Publication
(10) Pub. No.: US 2013/0196287 A1
(43) Pub. Date: Aug. 1, 2013

(54) DEVICE TO BE COUPLED TO A DENTAL IMPLANT ABUTMENT, PROVIDED WITH AT LEAST ONE CONNECTING ELEMENT

(71) Applicants: Silvano Umbero Tramonte, Milan (IT); Aldo Daniele Dominici, Milan (IT); Domenico Magnone, Milan (IT)

(72) Inventors: Silvano Umbero Tramonte, Milan (IT); Aldo Daniele Dominici, Milan (IT); Domenico Magnone, Milan (IT)

(21) Appl. No.: 13/539,129

(22) Filed: Jun. 29, 2012

(30) Foreign Application Priority Data
Jul. 6, 2011 (IT) MI2011 A001257

(51) Int. Cl. A61C 8/00; A61K 6/04

(52) U.S. Cl. CPC A61C 8/0069; A61K 6/04
USPC 433/173

(57) ABSTRACT

This invention refers to a device coupled to a dental implant abutment, where the so-called temporary teeth are mounted, provided with at least one connecting element, suitable to join it to at least another device coupled to a dental implant abutment, provided with at least one connecting element.

This application claims priority to Italian Application No. MI2011A001257, filed Jul. 6, 2011, which is incorporated by reference in its entirety.

### DESCRIPTION

1. Purpose of the Invention

The subject of this invention is a device coupled to a dental implant abutment, where the so-called temporary tooth is mounted. Such coupling device is provided with at least one connecting element suitable to be joined to at least one other connecting element.

2. State-of-the-Art

It is known how to connect with each other dental implants on which the so-called temporary teeth are mounted. Such implants are connected to each other right after their placement in the bone crest of the patient's mouth. Such implants must be connected with each other to facilitate the preservation of their position in the bone crest, up to the time when such bone crest has healed around the inserted dental implant. When the treated bone crest has healed, the connections are removed.

The known technique connects dental implants by welding a metal bar to the dental implants to be connected. The known connecting technique described above presents many disadvantages. As an example, not exhausting all possibilities, we indicate the following disadvantages:

(i) the effect of welding the bar to the implant abutment spoils the abutment itself, which has to be polished again when the said bar is removed;

(ii) the presence of the bar creates an unpleasant sensation of bulk for the patient;

(iii) there is a need to shape and passivate the bar;

(iv) during the welding action it is difficult to maintain the dental implant just inserted in the bony crest in a direction perpendicular to the occlusal plane. While welding the bar it is possible to compromise the perpendicular direction of one or more implants. In such case, the operator must unweld the bar to bring the implant back to an acceptable position, then repeat the welding operation;

(v) a long time is necessary to connect the bar to the dental implants, leading to operator and patient fatigue issues, as well as obvious hygiene problems.

### EXPLANATION OF THE INVENTION

This invention solves advantageously the issues of connecting the known dental implants by introducing a device coupled to the abutment of a dental implant, provided with at least one connecting element. Such coupling device has the advantage of being able to be installed on any dental implant in an easy and quick manner. Also, the current invention facilitates and makes quick and more hygienic the connection with another implant, preferably provided with the same coupling device. Such speed is due also to the ease of welding a connecting element, present on the coupling device mounted on the dental implant abutment, to the connecting device of another coupling element of another coupling device, subject of this invention, mounted on the abutment of another dental implant, thus obtaining the result of connecting two or more dental implants.

The subject of this invention also has the advantage of being able to be slipped over the abutment of any known dental implant.

In the preferred form of realization, the coupling device subject of this invention has:

(i) the shape of a hollow truncated cone;

(ii) the internal surface of the coupling device with longitudinal section has a female shape with respect to the dental implant. With respect to sub (ii), it has been verified with experiments that it is not necessary that the entire internal surface of the coupling device has a female shape with respect to the dental implant abutment, but it is sufficient that only the longitudinal section of the device has that shape.

The coupling device is provided with at least one connecting element located on the outside of such coupling device. This position facilitates the connection, particularly for welding purposes, with other contiguous dental implants, in particular with the connecting elements of the coupling devices slipped over the abutments of such implants. In the preferred version of realization of this invention, the connecting element can have any shape, but according to a series of experiments, the preferred shape of the connecting element is that of a small blade with rectangular surface.

With respect to the material to be used to realize the said connecting element, various types of materials have been subjected to experimental tests. According to the results of such tests, the materials that have given the best results are metals and their alloys. Among various metals and metal alloys, usually utilized in dentistry, tested in experiments, surprisingly optimal reactions to the welding process applied to join two connecting elements have been noticed in titanium or titanium alloys of grade lower than 4, in particular or titanium grade 2.

### FIGURE DESCRIPTION

FIG. 1 shows a three-dimensional view of some dental implants connected with the known technique.

FIG. 2 shows a two-dimensional view of the coupling device for the abutment of a dental implant, subject of this invention.

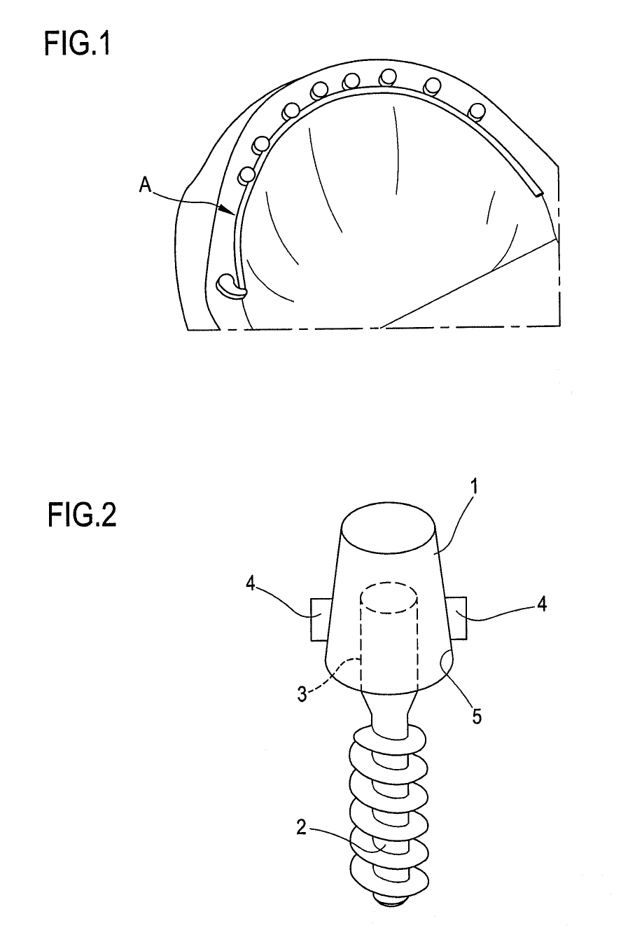

FIG. 1 shows a three-dimensional view of some dental implants connected with the known technique. In such a figure, the bulk represented by bar (A) used to connect the various dental implant abutments, is highlighted in particular.

With reference to FIG. 2, in a two-dimensional way have been represented: the coupling device (i) on the abutment of a dental implant (2) with a abutment (3) and two blade shaped connecting elements (4). Such coupling device (i) is suitable to be coupled with the abutment (3). Such coupling device (1) has the shape of a hollow truncated cone, with its internal surface (5) having the female longitudinal section with respect to the abutment (3) of the dental implant (2).

1. Coupling device (i) to the abutment of a dental implant (2), characterized by the fact of having a shape suitable to be slipped on the abutment (3) of the dental implant (2), and fitted with at least one connecting element (4) suitable to be joined to at least one more coupling device (i).

2. Coupling device (1) according to claim 1, characterized by the fact that such coupling device (1) has a hollow truncated conical shape.

3. Coupling device (1) according to claim 1, characterized by the fact that the internal surface (5) of such coupling device (1) has a female longitudinal section with respect to the abutment (3).

4. Coupling device (1) according to claim 1, characterized by the fact that the connecting element (4) is shaped as a small blade.

5. Coupling device (1) according to claim 1, characterized by the fact that the connecting element (4) is made of a metallic material suitable to be welded.

6. Coupling device (1) according to claim 5, characterized by the fact that the connecting element (4) is made of titanium.

7. Coupling device (1) according to claim 5, characterized by the fact that the connecting element (4) is made of a titanium alloy.
